(19) United States
(12) Patent Application Publication
(10) Pub. No.: US 2015/0011984 A1
(43) Pub. Date: Jan. 8, 2015

(54) CORNEAL VISUAL CENTER LOCALIZER (OR LOCATOR)

(71) Applicant: Mohammad Abdelfattah Daif, Zagazig (EG)

(72) Inventor: Mohammad Abdelfattah Daif, Zagazig (EG)

(21) Appl. No.: 14/373,434

(22) PCT Filed: Jun. 6, 2012
(86) PCT No.: PCT/EG2012/000020

§ 371 Date: Jul. 21, 2014

(30) Foreign Application Priority Data
Jan. 19, 2012 (AE) P56/2012
Jun. 6, 2012 (EG) 2012061013

(51) Int. Cl. A61F 9/008; A61B 19/00

(52) U.S. Cl. CPC A61F 9/008; A61B 19/5212; A61F 2009/00846; A61F 2009/00872; A61F 2009/00842; A61B 2019/5287
USPC 606/5

(57) ABSTRACT

Daifs Corneal Visual Center (CVC) Localizer (Locator) is a point Light Source of Infra Red (IR) light, attached and fixed to any eximer laser machine, located at equal distance and in exactly opposite direction from the middle of a detecting camera that detects IR light reflection from the corneal surface and the pupillary margin of the iris with a point fixation target or its virtual extension, perpendicular to the plan containing the localizer and the middle point the inlet of the detecting camera, exactly in the middle of the distance between the localizing point light source and the middle point the detecting camera. With the Localizer in position and activated, the CVC will be seen on the screen of IR eye tracking as a white dot somewhere inside the white circle that represents the detected pupillary margin. The center of the pupil is automatically detected by the eye tracking software system and is marked on the screen by a cross which will considered by treatment software of the machine as the center of the refraction correction laser treatment. After accurate in-focus positioning of eye to be treated with the patient fixing the fixation target, the position of this cross could be manually changed to the position of the white dot representing the CVC and confirming by OK button. The relation between the CVC and the pupillary margin image is automatically fixed by the eye tracking system all over the whole laser treatment of this particular eye.

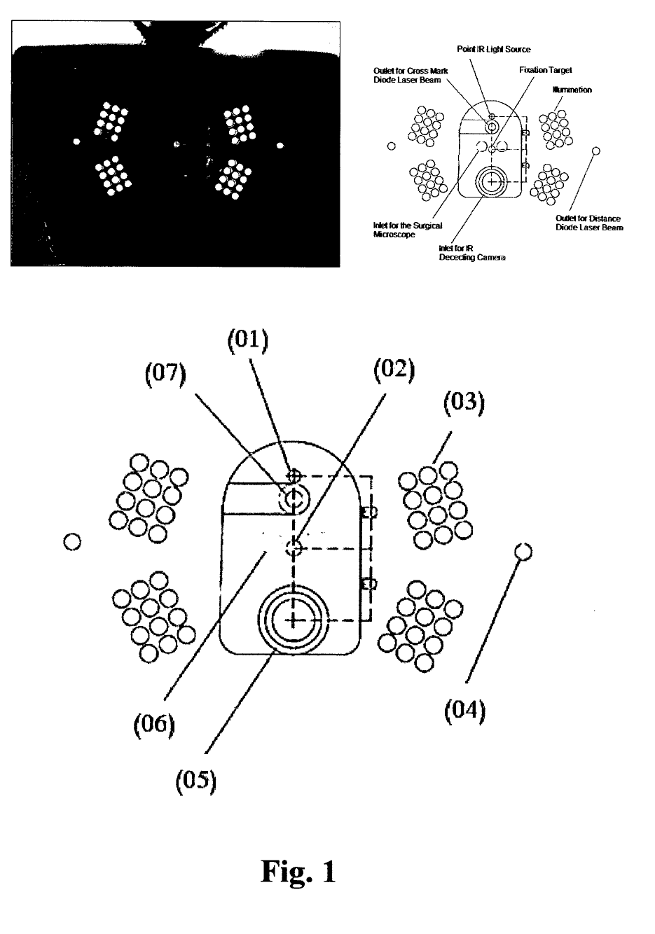

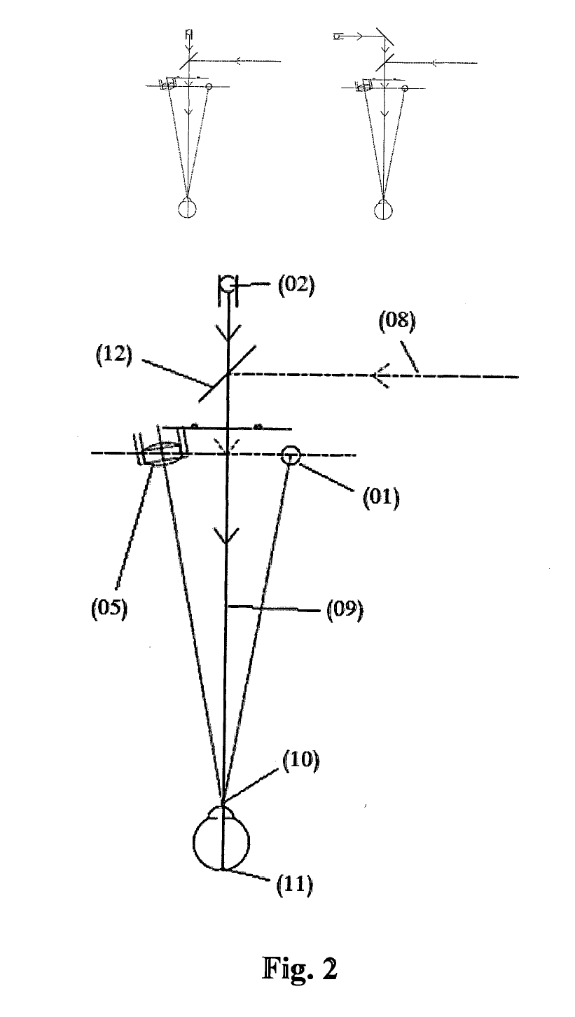

### TECHNICAL FIELD

Ophthalmology

### BACKGROUND ART

All the available eximer laser machines are designed to center the laser treatment for eye refraction correction on the corneal point perpendicular to the geometrical center of the pupil image detected behind the cornea during surgery. NO exceptions are currently available. This means that all laser refraction correction treatments are currently centered on the wrong point as the eye sight is centered on the visual axis of the eye (the virtual line between the retinal fovea, which is the retinal center, and the seen object or the object of regard) that crosses the cornea at a virtual point called the visual center of the cornea. The refraction of the eye measured by any of all the autorefractometer machines available is also centered on the visual axis of the eye which means that it is centered on the visual center of the cornea. The central papillary axis of the eye is the virtual line perpendicular to the center of the pupil. The continuation of the central pupillary axis crosses the cornea at a certain point. To this point the refraction correction laser treatment is centered and the distance on the cornea between it and the corneal visual center varies according to the angle between central papillary and visual axes of the eye which is called angle keppa. This angle keppa varies from one eye to another and accordingly the refraction corrective laser treatment is almost always laser treatment is almost always decentered away from the correct center, which is the visual center of the cornea, by some distance.

Trials were made to overcome this defect in the refraction correction laser treatments by making the treatment wavefront guided or topograghy guided or by using the iris details detection technology.

Unfortunately, the refraction correction part of the treatment is still centered on the pupil center in all cases and this decentration problem is not yet solved up till now.

However, most of the eximer laser machines have the option of manual change of the center of treatment and the surgeon can use it to change the center of treatment to a different point where he guesses that the corneal visual center is located. In fact, this feature is rarely used by the refractive surgeon as it is inaccurate and it may lead to a rather worse result if the guessing was in the wrong direction.

### PROBLEM AND DEFECT IN THE PRIOR ART

The effect of the previously described laser refraction correction treatment decentration shows postoperatively in the form decreased treatment accuracy and some residual astigmatism. Other effects are some increase in high order aberrations and some prismatic effect.

These effects are higher on treating higher errors and the effect of this decentration is more annoying for the treated hyperopic patients than myopic ones. This is because the rate of change of refraction from one point to the next as we go from the center of the treatment area towards the corneal visual center is much more rapid in treated hyperopic eyes than in treated myopic ones. That is why it is advisable on treating hyperopic eyes to increase the optic zone diameter from 6.5 mm to 7 mm. This relatively decreases the effect of decentration as it decreases the rate of change of refraction from the treatment center towards the corneal visual center.

### NEW IN THE INVENTION

The invention is a device to be attached and fixed to the eximer laser machine for accurate detection and localization of the corneal visual center in relation to the detected pupillary margin in the background so that the refraction correction laser treatment center could manually (software automatic detection could be done later) changed to be centered on the detected corneal visual center in relation to the detected papillary margin in the background. This relation is kept allover that laser treatment of this particular eye. So, the laser refraction correction treatment could be ACCURATELY CENTERED ON the correct center which is THE CORNEAL VISUAL CENTER, with all its advantages, and NOT on the papillary geometrical center which is the center for current treatments.

### DISCLOSURE OF INVENTION

Infrared (IR) spot light source (Corneal Visual Center Localizer or CVC Localizer) is attached and fixed to the eximer laser machine in an accurate positioning in relation to the IR detecting camera and the fixation target so that the virtual line in the horizontal plan that connect the CVC Localizer and the center of the Lens of the IR detecting camera is crossed at its exact middle (divided into two equal parts) by the visual line of the eye to be treated which is the virtual line between the fixation target and the retinal center (fovea) that crosses the cornea at the corneal visual center (CVC). In the machine, it is the virtual perpendicular line to the horizontal plan that goes exactly downwards from the fixation target.

When the patient lies down in the surgical supine position and fixes the fixation target with the eye to be treated, the corneal visual center (CVC) of the to be treated will be facing the fixation target and it will be the highest point in the cornea. The distance between the fixation target and the CVC is adjusted by up and down movements of the patient's surgical bed that leads to up and down movements of the eye to treated. The level of the eye is adjusted with aid of two red spots of diode laser. When two spots overlap to become one spot, it means that the cornea is in the proper position for laser treatment and in focus for the surgical microscope and in the proper position for the IR detecting camera that detects the pupillary margin for eye-tracking and treatment centration.

With the Daifs CVC Localizer in position and activated, the CVC will be seen on the screen of IR eye tracking as a white dot somewhere inside the white circle that represents the detected papillary margin. The center of the pupil is automatically detected by the eye tracking software system and is marked on the screen by a cross which will considered by treatment software of the machine as the center of the refraction correction laser treatment. The position of this cross could be manually changed to the position of the white dot representing the CVC and the relation between it and the pupillary margin image is automatically fixed by the eye tracking system all over the whole laser treatment of this particular eye.

Software automatic detection for CVC after correct and accurate eye positioning and confirming by pressing certain button could be developed and programmed easily later.

Brief Description of Drawings:

| | |
|---|---|
| 1—Point IR Light Source (Daif's CVC Localizer). | 2—Fixation Target. |
| 3—Illumination. | 4—Outlet of |
| | Distance Diode |
| | Laser. |
| 5—Inlet of IR Detecting Camera. | 6—Inlet of Surgical |
| | Microscope. |
| 7—Outlet of Cross Mark Diode Laser. | 8—Eximer Laser. |
| 9—Visual Axis. | 10—Corneal Visual |
| | Center (CVC). |
| 11—Fovea. | 12—Glass Mirror. |

N.B.:
The Eye, Glass Mirror and Eximer Laser Beam (in Drawing No. 2) had been rotated 90° clockwise around the Vertical Axis for Simplicity.

### HOW TO USE THE INVENTION

With the Daif's CVC Localizer in position and activated, the CVC will be seen on the screen of IR eye tracking as a white dot somewhere inside the white circle that represents the detected papillary margin. The center of the pupil is automatically detected by the eye tracking software system and is marked on the screen by a cross which will considered by treatment software of the machine as the center of the refraction correction laser treatment. After accurate in-focus positioning of eye to be treated with the patient fixing the fixation target, the position of this cross could be manually changed to the position of the white dot representing the CVC and confirming by OK button. The relation between the CVC and the pupillary margin image is automatically fixed by the eye tracking system all over the whole laser treatment of this particular eye.

Software automatic detection for CVC after correct and accurate eye positioning and confirming by pressing certain button could be developed and programmed easily later.

1. The nomenclature: Corneal Visual Center Localizer or CVC Localizer or Locator.

2. A real or virtual (image or reflected or refracted) point Light Source of Infra Red (IR), visible or other invisible light attached and fixed any eximer laser machine or any ophthalmic diagnostic or therapeutic machine or device (or any similar device) located at equal distance and in exactly opposite direction from the middle of a detecting appropriate camera (that can detect its invisible or visible light reflection from the corneal surface as well as the pupillary margin of the iris) or the middle of a detecting inlet of a surgical microscope with a point fixation target or its virtual extension, perpendicular to the plan containing the localizing point light source and the middle point the inlet of a detecting appropriate camera, exactly in the middle of the distance between the localizing point light source and the middle point the detecting appropriate camera.
